(19) United States
(12) Patent Application Publication
(10) Pub. No.: US 2007/0207455 A1
(43) Pub. Date: Sep. 6, 2007

(54) Compositions and methods for detecting an HCV-1 subtype

(75) Inventors: Scott M. Law, Madison, WI (US); Vecheslav A. Elagin, Waunakee, WI (US)

Correspondence Address: Medlen & Carroll, LLP;Suite 350, 101 Howard Street, San Francisco, CA, 94105, US

(73) Assignee: Third Wave Technologies, Inc., Madison, WI (US)

(21) Appl. No.: 11/601,551

(22) Filed: Nov. 17, 2006

Related U.S. Application Data
(60) Provisional application No. 60/737,656, filed on Nov. 17, 2005.

(51) Int. Cl. C12Q 1/70; C12Q 1/68

(52) U.S. Cl. 435/5; 435/6

(57) ABSTRACT

The present invention provides methods and compositions for detecting hepatitis C virus (HCV). In particular, the present invention provides nucleic acid detection assays configured to detect a novel sub-type of HCV-1.

The present application claims priority to U.S. Provisional Application 60/737,656 filed Nov. 17, 2005, which is herein incorporated by reference.

### FIELD OF THE INVENTION

The present invention provides methods and compositions for detecting hepatitis C virus (HCV). In particular, the present invention provides nucleic acid detection assays configured to detect a novel subtype of HCV-1.

### BACKGROUND

Hepatitis C Virus (HCV) accounts for nearly all cases of non-A, non-B hepatitis (NANBH) (Choo, Q.-L., et al., Proc. Natl. Acad. Sci. USA 88: 2451-2455 (1988)) and is a persistent health threat worldwide, with more than one million new cases reported annually (Zein, N. N. Clin. Micro. Rev. 13: 223-235 (2000)). HCV infection is almost always chronic and persistent. The most severe consequences of HCV infection are chronic liver disease and death, and HCV infection is the primary impetus for liver transplantation in the US (Zein, supra).

HCV is a positive strand single-stranded RNA virus approximately 10 kb long belonging to the Flaviviridae family (Zein, supra). There is considerable heterogeneity among isolates found in different geographic regions. These differences have been classified into multiple genotypes and subtypes. Although various different criteria have been used to characterize these genotypes, two principal modes of classification have been adopted. The more widely used of these was created by Peter Simmonds and uses Arabic numerals to denote different genotypes and latin letters for subtypes, e.g. type 1a, 1b, 2a, etc. (reviewed in Simmonds, P. Hepatology. February;21(2):570-83 (1995) and Simmonds, P. J. Hepatol.; 31 Suppl 1:54-60 (1999)). According to this system, genotypes 1-3 are the prevalent types found in North America, Europe, and Japan, and the remaining types are found at various frequencies in parts of Asia and Africa. Thus in some instances HCV genotype and subtype may be of epidemiological importance, for example in determining the etiology of infection.

Efforts have been undertaken to elucidate the clinical significance of different genotypes and subtypes. Some studies suggest that infections of type 1, in particular type 1b, may be associated with more severe disease and earlier recurrence (Zein, N. N. et al., Liver Transplant. Surg. 1: 354-357 (1995); Gordon et al., Transplantation 63: 1419-1423 (1997)). Certain studies have also indicated that genotypes other than type 1 (e.g. 1a or 1b) may respond more favorably to various treatments, e.g. interferon (McHutchison, J. G., et al., N. Engl. J. Med., 339: 1485-1492 (1998)). It has been suggested that determination of HCV genotype in combination with other diagnostic markers, such as viral load, may be of value in arriving at disease prognoses (Zein, N. N. supra), and determining the course of treatment (National Institutes of Health Consensus Development Conference Statement; Management of Hepatitis C: 2002; Jun. 10-11, 2002).

Different regions of the HCV genome have been used to determine genotype. The HCV genome includes relatively conserved regions, such as the 5′ and 3′ untranslated regions (UTR), variable regions (e.g. E1 and non-structural (NS) 5B), as well as hypervariable regions such as those encoding the envelope proteins (Halfon, P. CLI, April 2002). Studies have been carried out to correlate the presence of particular sequences in the conserved regions with sequences in the variable regions, in particular the NS-5B (Stuyver, L., et al., J. Clin. Micro., 34: 2259-2266 (1996)). As a result of such studies, genotyping assays based on conserved regions, particularly the 5′ UTR, have been developed to simplify the task of identifying which viral type or types are present in a specimen. Given the existence of commercially available viral load assays that rely on amplifying all or part of the 5′ UTR, the ability to determine HCV genotype based on discrete sequence differences in this conserved region presents a convenient means of obtaining extensive diagnostic information from a single amplified nucleic acid, e.g. a RT-PCR or Transcription Mediated Amplification (TMA) amplicon.

Various molecular biological methods have been applied to the task of determining HCV genotype using the 5′ UTR. These include reverse dot-blot analysis (e.g. Inno LIPA, Innogenetics, Ghent, Belgium, as described in Stuyver, L. et al., J Clin Microbiol. 1996 September; 34(9):2259-66, and U.S. Pat. No. 6,495,670 both of which are herein incorporated by reference); direct DNA sequencing, such as TRUEGENE HCV 5′NC genotyping kit, Bayer Diagnostics, Berkeley, Calif. (e.g., as described in Germer, J. J. et al. J Clin Microbiol. 2003 October; 41(10):4855-7, herein incorporated by reference), and pyrosequencing (Pyrosequencing AB, Uppsala, Sweden, as described in U.S. Pat. No. 6,258,568, herein incorporated by reference).

In addition to these molecular methods, serological methods for determining genotype have been introduced, e.g. the RIBA SIA test (Chiron Corp., Emeryville, Calif.) and the Murex HCV serotyping enzyme immune assay (Murex Diagnostics Ltd, Dartford, UK). Some studies indicate that serologic typing may be limited in terms of specificity and sensitivity (Zein, supra)

Therefore, there exists a need for a rapid, sensitive, accurate, and homogeneous method for accurately determining HCV genotypes and sub-types in a clinical sample, e.g. blood or blood fraction, such that the proper treatment regimen be provided to an infected subject.

### SUMMARY OF THE INVENTION

The present invention provides methods and compositions for detecting hepatitis C virus (HCV). In particular, the present invention provides nucleic acid detection assays configured to detect a novel subtype of HCV-1. The novel HCV-1 subtype can be referred to as HCV-1twt. HCV-1twt contains an adenine at position −166 and a guanidine at position −119 as numbered in FIG. 1. Part of the 5′ UTR sequence of HCV-1twt is shown as SEQ ID NO:6 in FIG. 1.

In some embodiments, the present invention provides compositions comprising a first nucleic acid detection assay, wherein the first nucleic acid detection assay comprises a reagent configured to specifically detect the presence of an adenine at position −166 of the 5′ untranslated region of a hepatitis C virus (using the standard base numbering in the art, which is shown in FIG. 1). In certain embodiments, the first nucleic acid detection assay comprises an invasive cleavage detection assay (e.g. an INVADER nucleic acid detection assay). In other embodiments, the first nucleic acid detection assay comprises INVADER assay reagents. In particular embodiments, the first nucleic acid detection assay is selected from the group consisting of: a TAQMAN assay, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, and a sandwich hybridization assay.

In additional embodiments, the compositions further comprise a second nucleic acid detection assay configured to specifically detect at least one of the following positions in the 5′ untranslated region of hepatitis C virus: adenine at position −163; cytosine, guanidine, or thymine at position −159; cytosine at position −155; guanidine at position −132; adenine at position −128; thymine at position −122; guanidine or adenine at position −119; guanidine at position −118, thymine at position −80; and cytosine at position −72. In particular embodiments, the first nucleic acid detection assay and the second nucleic acid detection assay together are capable of identifying the hepatitis C virus as genotype 1, specifically HCV-1twt.

In some embodiments, the present invention provides compositions comprising a nucleic acid detection assay, wherein the nucleic acid detection assay comprises a reagent configured to specifically detect the presence of a guanidine at position −119 of the 5′ untranslated region of a hepatitis C virus. In certain embodiments, the nucleic acid detection assay comprises an invasive cleavage detection assay (e.g. an INVADER nucleic acid detection assay). In other embodiments, the nucleic acid detection assay comprises INVADER assay reagents.

In particular embodiments, the present invention provides compositions comprising a nucleic acid detection assay that is configured to detect the presence or absence of both a guanidine at position −119 and an adenine at position −166 of the 5′ untranslated region of a hepatitis C virus.

In particular embodiments, the nucleic acid detection assay is selected from the group consisting of: a TAQMAN assay, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, a sandwich hybridization assay, a Line Probe Assay (LiPA, U.S. Pat. No. 5,846,704) and TMA-LiPA (Bayer). In other embodiments, the nucleic acid detection assay is configured to detect the presence of a guanidine at position −119 of the hepatitis C virus. In additional embodiments, the nucleic acid detection assay is capable of identifying the hepatitis C virus as HCV-1twt (e.g. without the need for detecting bases at other positions).

In some embodiments, the present invention provides methods comprising contacting a sample suspected of containing hepatitis C virus with a first nucleic acid detection assay under conditions such that the presence or absence of adenine at position −166 of the 5′ untranslated region of the hepatitis C virus is detected. In other embodiments, the first nucleic acid detection assay comprises an invasive cleavage detection assay (e.g. an INVADER nucleic acid detection assay). In certain embodiments, the first nucleic acid detection assay is selected from the group consisting of: a TAQMAN assay, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, a sandwich hybridization assay, a Line Probe Assay (LiPA, U.S. Pat. No. 5,846,704) and TMA-LiPA (Bayer).

In additional embodiments, the method further comprises contacting the sample with a second nucleic acid detection assay configured to detect at least one of the following positions in the 5′ untranslated region of HCV: adenine at position −163; cytosine, guanidine, or thymine at position −159; cytosine at position −155; guanidine at position −132; adenine at position −128; thymine at position −122; guanidine or adenine at position −119; guanidine at position −118, thymine at position −80; and cytosine at position −72.

In particular embodiments, the first nucleic acid detection assay and the second nucleic acid detection assay together are capable of identifying the hepatitis C virus as HCV-1twt. In some embodiments, the methods further comprise the step of selecting a therapy for a subject (e.g., selecting an appropriate drug, selecting an appropriate dose of drug, avoiding certain drugs, continuing administration of a certain drug for a certain number of days, etc.) based on the identification of HCV-1twt in the sample.

In certain embodiments, the present invention provides methods comprising contacting a sample suspected of containing hepatitis C virus with a nucleic acid detection assay under conditions such that the presence or absence of a guanidine at position −119 of the 5′ untranslated region of the hepatitis C virus is detected. In particular embodiments, the nucleic acid detection assay comprises an invasive cleavage detection assay. In some embodiments, the nucleic acid detection assay is selected from the group consisting of: a TAQMAN assay, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, a sandwich hybridization assay, a Line Probe Assay (LiPA, U.S. Pat. No. 5,846,704) and TMA-LiPA (Bayer). In additional embodiments, the sample if from a subject (e.g. human).

In other embodiments, the nucleic acid detection assay is capable of identifying the hepatitis C virus as HCV-1twt. In certain embodiments, the methods further comprise the step of selecting a therapy for a subject (e.g., selecting an appropriate drug, selecting an appropriate dose of drug, avoiding certain drugs, continuing administration of a certain drug for a certain number of days, etc.) based on the identification of HCV-1twt in the sample.

In some embodiments, the present invention provides a nucleic acid detection assay kit for detecting an adenine at position −166 of the untranslated region of a hepatitis C virus, the kit comprising; a) a first component comprising a nucleic acid sequence configured to hybridize to the untranslated region of the hepatitis C virus; and b) a second component comprising an enzyme, wherein the enzyme comprises a polymerase or structure-specific nuclease.

In other embodiments, the present invention provides nucleic acid detection assay kits for detecting a guanidine at position −119 of the untranslated region of a hepatitis C virus, the kit comprising; a) a first component comprising a nucleic acid sequence configured to hybridize to the untranslated region of the hepatitis C virus; and b) a second component comprising an enzyme, wherein the enzyme comprises a polymerase or structure specific nuclease.

In additional embodiments, the present invention provides compositions comprising a first nucleic acid sequence or a second nucleic acid sequence, wherein the first nucleic acid sequence comprises at least 12 contiguous bases from SEQ ID NO:6 and includes the adenine at position −166 of SEQ ID NO:6 as numbered in FIG. 1, and wherein the second nucleic acid sequence is configured to hybridize to the first nucleic acid sequence under high stringency conditions. In some embodiments, the first and second nucleic acid sequences are both present in the composition. In other embodiments, the first nucleic acid sequence comprises at least 13, 14, 15, 16, 17, 18, 19, 20 or 25 bases from SEQ ID NO:6. In further embodiments, the kits further comprise additional detection assay reagents, including, but not limited to, polymerases, enzymes (e.g. structure specific enzymes), buffers, instructions for kit use, etc. In particular embodiments, the kits provide nucleic acid sequences configured to bind specifically with HCV-1twt (e.g. configured to only bind HCV-1twt and not not HCV sequences, and/or configured to not bind other sequences potentially present in a blood sample, such as herpes virus or human genomic DNA).

In additional embodiments, the present invention provides compositions comprising an isolated first nucleic acid sequence or an isolated second nucleic acid sequence, wherein the first nucleic acid sequence comprises at least 12 contiguous bases from SEQ ID NO:6 and includes the guanidine at position −119 of SEQ ID NO:6 as numbered in FIG. 1, and wherein the second nucleic acid sequence is configured to hybridize to the first nucleic acid sequence under high stringency conditions. In certain embodiments, the first and second nucleic acid sequences are both present in the composition. In other embodiments, the first nucleic acid sequence comprises at least 13, 14, 15, 16, 17, 18, 19, 20 or 25 bases from SEQ ID NO:6. In further embodiments, the kits further comprise additional detection assay reagents, including, but not limited to, polymerases, enzymes (e.g. structure specific enzymes), buffers, instructions for kit use, etc. In particular embodiments, the kits provide nucleic acid sequences configured to bind specifically with HCV-1twt (e.g. configured to only bind HCV-1twt and not not HCV sequences, and/or configured to not bind other sequences potentially present in a blood sample, such as herpes virus or human genomic DNA).

In some embodiments, the present invention provides compositions comprising an isolated first nucleic acid sequence or an isolated second nucleic acid sequence, wherein the first nucleic acid sequence comprises a sequence selected from the group consisting of SEQ ID NOs:7-21 and SEQ ID NOs:22-37, and wherein the second nucleic acid sequence is configured to hybridize to the first nucleic acid sequence under high stringency conditions. In certain embodiments, the first and second nucleic acid sequences are both present in the composition.

The method of the present invention are not limited by the nature of the 5′ UTR HCV target nucleic acid. In some embodiments, the target nucleic acid is single stranded or double stranded DNA or RNA. In some embodiments, double stranded nucleic acid is rendered single stranded (e.g., by heat) prior to contact with a nucleic acid detection assay. In some embodiments, the source of target nucleic acid comprises a sample containing genomic RNA. Samples include, but are not limited to, tissue sections, blood, blood fractions (e.g. plasma, serum) saliva, cerebral spinal fluid, pleural fluid, milk, lymph, sputum and semen.

In some embodiments, the target nucleic acid comprises genomic RNA. In other embodiments, the target nucleic acid comprises synthetic DNA or RNA. In some preferred embodiments, synthetic DNA or RNA within a sample is created using a purified polymerase. In some embodiments, creation of synthetic DNA using a purified polymerase comprises the use of PCR. In other embodiments, the synthetic DNA created comprises all or a portion of the 5′UTR of the HCV genome. In certain embodiments, creation of synthetic DNA is accomplished by using a purified reverse transcriptase to generate a cDNA prior to PCR. In some embodiments such RT-PCR is carried out with commercial kits such as COBAS AMPLICOR or COBAS TAQMAN (Roche Molecular Systems).

The HCV nucleic acid detection assays provided in the present invention may include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884, 6,183,960 and 6,235,502, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci. USA 88, 189-93 (1991)); and sandwich hybridization methods (e.g., U.S. Pat. No. 5,288,609, herein incorporated by reference in its entirety).

In other embodiments, the nucleic acid detection assays comprise first and second oligonucleotides configured to form an invasive cleavage structure (e.g. an INVADER assay) in combination with an HCV 5′ UTR target sequence. In particular embodiments, the first oligonucleotide comprises a 5′ portion and a 3′ portion, wherein the 3′ portion is configured to hybridize to the target sequence, and wherein the 5′ portion is configured to not hybridize to the target sequence. In other embodiments, the second oligonucleotide comprises a 5′ portion and a 3′ portion, wherein the 5′ portion is configured to hybridize to the target sequence, and wherein the 3′ portion is configured to not hybridize to the target sequence.

Definitions

To facilitate an understanding of the present invention, a number of terms and phrases are defined below:

As used herein, the terms “subject” and “patient” refer to any organism including plants, microorganisms and animals (e.g., mammals such as dogs, cats, livestock, and humans).

As used herein, the term “INVADER assay reagents” refers to one or more reagents for detecting target sequences (e.g. HCV 5′ UTR sequences), said reagents comprising oligonucleotides capable of forming an invasive cleavage structure in the presence of the target sequence. In some embodiments, the INVADER assay reagents further comprise an agent for detecting the presence of an invasive cleavage structure (e.g., a cleavage agent). In certain embodiments, the oligonucleotides comprise first and second oligonucleotides, said first oligonucleotide comprising a 5′ portion complementary to a first region of the target nucleic acid and said second oligonucleotide comprising a 3′ portion and a 5′ portion, said 5′ portion complementary to a second region of the target nucleic acid downstream of and contiguous to the first portion. In some embodiments, the 3′ portion of the second oligonucleotide comprises a 3′ terminal nucleotide not complementary to the target nucleic acid. In preferred embodiments, the 3′ portion of the second oligonucleotide consists of a single nucleotide not complementary to the target nucleic acid.

In particular embodiments, INVADER assay reagents are configured to detect a target nucleic acid sequence comprising first and second non-contiguous single-stranded regions separated by an intervening region comprising a double-stranded region. In some embodiments, the INVADER assay reagents comprise a bridging oligonucleotide capable of binding to said first and second non-contiguous single-stranded regions of a target nucleic acid sequence. In particularly preferred embodiments, either or both of said first or said second oligonucleotides of said INVADER assay reagents are bridging oligonucleotides.

In some embodiments, the INVADER assay reagents further comprise a solid support. For example, in some embodiments, the one or more oligonucleotides of the assay reagents (e.g., first and/or second oligonucleotide, whether bridging or non-bridging) is attached to said solid support. In some embodiments, the INVADER assay reagents further comprise a buffer solution. In some preferred embodiments, the buffer solution comprises a source of divalent cations (e.g., Mn²⁺ and/or Mg²⁺ ions). Individual ingredients (e.g., oligonucleotides, enzymes, buffers, target nucleic acids) that collectively make up INVADER assay reagents are termed “INVADER assay reagent components”.

In some embodiments, the INVADER assay reagents further comprise a third oligonucleotide complementary to a third portion of the target nucleic acid upstream of the first portion of the first target nucleic acid. In yet other embodiments, the INVADER assay reagents further comprise a target nucleic acid. In some embodiments, the INVADER assay reagents further comprise a second target nucleic acid. In yet other embodiments, the INVADER assay reagents further comprise a third oligonucleotide comprising a 5′ portion complementary to a first region of the second target nucleic acid. In some specific embodiments, the 3′ portion of the third oligonucleotide is covalently linked to the second target nucleic acid. In other specific embodiments, the second target nucleic acid further comprises a 5′ portion, wherein the 5′ portion of the second target nucleic acid is the third oligonucleotide. In still other embodiments, the INVADER assay reagents further comprise an ARRESTOR molecule (e.g., ARRESTOR oligonucleotide).

In some preferred embodiments, the INVADER assay reagents further comprise reagents for detecting a nucleic acid cleavage product. In some embodiments, one or more oligonucleotides in the INVADER assay reagents comprise a label. In some preferred embodiments, said first oligonucleotide comprises a label. In other preferred embodiments, said third oligonucleotide comprises a label. In particularly preferred embodiments, the reagents comprise a first and/or a third oligonucleotide labeled with moieties that produce a fluorescence resonance energy transfer (FRET) effect.

In some embodiments one or more the INVADER assay reagents may be provided in a predispensed format (i.e., premeasured for use in a step of the procedure without re-measurement or re-dispensing). In some embodiments, selected INVADER assay reagent components are mixed and predispensed together. In other embodiments, In preferred embodiments, predispensed assay reagent components are predispensed and are provided in a reaction vessel (including but not limited to a reaction tube or a well, as in, e.g., a microtiter plate). In particularly preferred embodiments, predispensed INVADER assay reagent components are dried down (e.g., desiccated or lyophilized) in a reaction vessel.

In some embodiments, the INVADER assay reagents are provided as a kit. As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials.

In some embodiments, the present invention provides INVADER assay reagent kits, or other nucleic acid detection assay kits, comprising one or more of the components necessary for practicing the present invention. For example, the present invention provides kits for storing or delivering the enzymes and/or the reaction components necessary to practice an INVADER assay. The kit may include any and all components necessary or desired for assays including, but not limited to, the reagents themselves, buffers, control reagents (e.g., tissue samples, positive and negative control target oligonucleotides, etc.), solid supports, labels, written and/or pictorial instructions and product information, inhibitors, labeling and/or detection reagents, package environmental controls (e.g., ice, desiccants, etc.), and the like. In some embodiments, the kits provide a sub-set of the required components, wherein it is expected that the user will supply the remaining components. In some embodiments, the kits comprise two or more separate containers wherein each container houses a subset of the components to be delivered. For example, a first container (e.g., box) may contain an enzyme (e.g., structure specific cleavage enzyme in a suitable storage buffer and container), while a second box may contain oligonucleotides (e.g., INVADER oligonucleotides, probe oligonucleotides, control target oligonucleotides, etc.).

The term “label” as used herein refers to any atom or molecule that can be used to provide a detectable (preferably quantifiable) effect, and that can be attached to a nucleic acid or protein. Labels include but are not limited to dyes; radiolabels such as ³²P; binding moieties such as biotin; haptens such as digoxygenin; luminogenic, phosphorescent or fluorogenic moieties; mass tags; and fluorescent dyes alone or in combination with moieties that can suppress (“quench”) or shift emission spectra by fluorescence resonance energy transfer (FRET). FRET is a distance-dependent interaction between the electronic excited states of two molecules (e.g., two dye molecules, or a dye molecule and a non-fluorescing quencher molecule) in which excitation is transferred from a donor molecule to an acceptor molecule without emission of a photon. (Stryer et al., 1978, Ann. Rev. Biochem., 47:819; Selvin, 1995, Methods Enzymol., 246:300, each incorporated herein by reference). As used herein, the term “donor” refers to a fluorophore that absorbs at a first wavelength and emits at a second, longer wavelength. The term “acceptor” refers to a moiety such as a fluorophore, chromophore, or quencher that has an absorption spectrum that overlaps the donor's emission spectrum, and that is able to absorb some or most of the emitted energy from the donor when it is near the donor group (typically between 1-100 nm). If the acceptor is a fluorophore, it generally then re-emits at a third, still longer wavelength; if it is a chromophore or quencher, it then releases the energy absorbed from the donor without emitting a photon. In some embodiments, changes in detectable emission from a donor dye (e.g. when an acceptor moiety is near or distant) are detected. In some embodiments, changes in detectable emission from an acceptor dye are detected. In preferred embodiments, the emission spectrum of the acceptor dye is distinct from the emission spectrum of the donor dye such that emissions from the dyes can be differentiated (e.g., spectrally resolved) from each other.

In some embodiments, a donor dye is used in combination with multiple acceptor moieties. In a preferred embodiment, a donor dye is used in combination with a non-fluorescing quencher and with an acceptor dye, such that when the donor dye is close to the quencher, its excitation is transferred to the quencher rather than the acceptor dye, and when the quencher is removed (e.g., by cleavage of a probe), donor dye excitation is transferred to an acceptor dye. In particularly preferred embodiments, emission from the acceptor dye is detected. See, e.g., Tyagi, et al., Nature Biotechnology 18:1191 (2000), which is incorporated herein by reference.

Labels may provide signals detectable by fluorescence (e.g., simple fluorescence, FRET, time-resolved fluorescence, fluorescence polarization, etc.), radioactivity, colorimetry, gravimetry, X-ray diffraction or absorption, magnetism, enzymatic activity, characteristics of mass or behavior affected by mass (e.g., MALDI time-of-flight mass spectrometry), and the like. A label may be a charged moiety (positive or negative charge) or alternatively, may be charge neutral. Labels can include or consist of nucleic acid or protein sequence, so long as the sequence comprising the label is detectable.

As used herein, the term “distinct” in reference to signals refers to signals that can be differentiated one from another, e.g., by spectral properties such as fluorescence emission wavelength, color, absorbance, mass, size, fluorescence polarization properties, charge, etc., or by capability of interaction with another moiety, such as with a chemical reagent, an enzyme, an antibody, etc.

As used herein, the terms “complementary” or “complementarity” are used in reference to polynucleotides (i.e., a sequence of nucleotides such as an oligonucleotide or a target nucleic acid) related by the base-pairing rules. For example, for the sequence “5′-A-G-T-3′,” is complementary to the sequence “3′-T-C-A-5′.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection methods which depend upon binding between nucleic acids. Either term may also be used in reference to individual nucleotides, especially within the context of polynucleotides. For example, a particular nucleotide within an oligonucleotide may be noted for its complementarity, or lack thereof, to a nucleotide within another nucleic acid strand, in contrast or comparison to the complementarity between the rest of the oligonucleotide and the nucleic acid strand.

As used herein, the term “hybridization” is used in reference to the pairing of complementary nucleic acids. Hybridization and the strength of hybridization (i.e., the strength of the association between the nucleic acids) is influenced by such factors as the degree of complementary between the nucleic acids, stringency of the conditions involved, and the Tₘ of the formed hybrid. “Hybridization” methods involve the annealing of one nucleic acid to another, complementary nucleic acid, i.e., a nucleic acid having a complementary nucleotide sequence. The ability of two polymers of nucleic acid containing complementary sequences to find each other and anneal through base pairing interaction is a well-recognized phenomenon. The initial observations of the “hybridization” process by Marmur and Lane, Proc. Natl. Acad. Sci. USA 46:453 (1960) and Doty et al., Proc. Natl. Acad. Sci. USA 46:461 (1960) have been followed by the refinement of this process into an essential tool of modern biology.

The complement of a nucleic acid sequence as used herein refers to an oligonucleotide which, when aligned with the nucleic acid sequence such that the 5′ end of one sequence is paired with the 3′ end of the other, is in “antiparallel association.” Certain bases not commonly found in natural nucleic acids may be included in the nucleic acids of the present invention and include, for example, inosine and 7-deazaguanine. Complementarity need not be perfect; stable duplexes may contain mismatched base pairs or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs.

As used herein the term “stringency” is used in reference to the conditions of temperature, ionic strength, and the presence of other compounds such as organic solvents, under which nucleic acid hybridizations are conducted. Those skilled in the art will recognize that “stringency” conditions may be altered by varying the parameters just described either individually or in concert. With “high stringency” conditions, nucleic acid base pairing will occur only between nucleic acid fragments that have a high frequency of complementary base sequences (e.g., hybridization under “high stringency” conditions may occur between homologs with about 85-100% identity, preferably about 70-100% identity). With medium stringency conditions, nucleic acid base pairing will occur between nucleic acids with an intermediate frequency of complementary base sequences (e.g., hybridization under “medium stringency” conditions may occur between homologs with about 50-70% identity). Thus, conditions of “weak” or “low” stringency are often required with nucleic acids that are derived from organisms that are genetically diverse, as the frequency of complementary sequences is usually less.

“High stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 0.1×SSPE, 1.0% SDS at 42 C when a probe of about 500 nucleotides in length is employed.

“Medium stringency conditions” when used in reference to nucleic acid hybridization comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.5% SDS, 5× Denhardt's reagent and 100 μg/ml denatured salmon sperm DNA followed by washing in a solution comprising 1.0×SSPE, 1.0% SDS at 42 C when a probe of about 500 nucleotides in length is employed.

“Low stringency conditions” comprise conditions equivalent to binding or hybridization at 42 C in a solution consisting of 5×SSPE (43.8 g/l NaCl, 6.9 g/l NaH₂PO₄H₂O and 1.85 g/l EDTA, pH adjusted to 7.4 with NaOH), 0.1% SDS, 5× Denhardt's reagent [50× Denhardt's contains per 500 ml: 5 g Ficoll (Type 400, Pharamcia), 5 g BSA (Fraction V; Sigma)] and 100 g/ml denatured salmon sperm DNA followed by washing in a solution comprising 5×SSPE, 0.1% SDS at 42 C when a probe of about 500 nucleotides in length is employed.

As used herein, the term “Tₘ” is used in reference to the “melting temperature.” The melting temperature is the temperature at which a population of double-stranded nucleic acid molecules becomes half dissociated into single strands. Several equations for calculating the Tₘ of nucleic acids are well known in the art. As indicated by standard references, a simple estimate of the Tₘ value may be calculated by the equation: Tₘ=81.5+0.41(% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr. Thermodynamics and NMR of internal G. T mismatches in DNA. Biochemistry 36, 10581-94 (1997) include more sophisticated computations which take structural and environmental, as well as sequence characteristics into account for the calculation of Tₘ.

The term “oligonucleotide” as used herein is defined as a molecule comprising two or more deoxyribonucleotides or ribonucleotides, at least 5 nucleotides, for example at least about 10-30 nucleotides, although longer oligonucleotides (e.g. 50 . . . 100 . . . , etc.) are contemplated. The exact size will depend on many factors, which in turn depend on the ultimate function or use of the oligonucleotide. The oligonucleotide may be generated in any manner, including chemical synthesis, DNA replication, reverse transcription, PCR, or a combination thereof.

Because mononucleotides are reacted to make oligonucleotides in a manner such that the 5′ phosphate of one mononucleotide pentose ring is attached to the 3′ oxygen of its neighbor in one direction via a phosphodiester linkage, an end of an oligonucleotide is referred to as the “5′end” if its 5′ phosphate is not linked to the 3′ oxygen of a mononucleotide pentose ring and as the “3′end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. A first region along a nucleic acid strand is said to be upstream of another region if the 3′ end of the first region is before the 5′ end of the second region when moving along a strand of nucleic acid in a 5′ to 3′ direction.

When two different, non-overlapping oligonucleotides anneal to different regions of the same linear complementary nucleic acid sequence, and the 3′ end of one oligonucleotide points towards the 5′ end of the other, the former may be called the “upstream” oligonucleotide and the latter the “downstream” oligonucleotide. Similarly, when two overlapping oligonucleotides are hybridized to the same linear complementary nucleic acid sequence, with the first oligonucleotide positioned such that its 5′ end is upstream of the 5′ end of the second oligonucleotide, and the 3′ end of the first oligonucleotide is upstream of the 3′ end of the second oligonucleotide, the first oligonucleotide may be called the “upstream” oligonucleotide and the second oligonucleotide may be called the “downstream” oligonucleotide.

The term “primer” refers to an oligonucleotide that is capable of acting as a point of initiation of synthesis when placed under conditions in which primer extension is initiated. An oligonucleotide “primer” may occur naturally, as in a purified restriction digest or may be produced synthetically.

The term “cleavage structure” as used herein, refers to a structure that is formed by the interaction of at least one probe oligonucleotide and a target nucleic acid, forming a structure comprising a duplex, the resulting structure being cleavable by a cleavage agent, including but not limited to an enzyme. The cleavage structure is a substrate for specific cleavage by the cleavage agents in contrast to a nucleic acid molecule that is a substrate for non-specific cleavage by agents such as phosphodiesterases which cleave nucleic acid molecules without regard to secondary structure (i.e., no formation of a duplexed structure is required).

The term “cleavage agent” as used herein refers to any agent that is capable of cleaving a cleavage structure, including but not limited to enzymes. “Structure-specific nucleases” or “structure-specific enzymes” are enzymes that recognize specific secondary structures in a nucleic molecule and cleave these structures. The cleavage agents of the invention cleave a nucleic acid molecule in response to the formation of cleavage structures; it is not necessary that the cleavage agents cleave the cleavage structure at any particular location within the cleavage structure.

The cleavage agent may include nuclease activity provided from a variety of sources including the Cleavase enzymes, the FEN-1 endonucleases (including RAD2 and XPG proteins), Taq DNA polymerase and E. coli DNA polymerase I. The agent may include enzymes having 5′ nuclease activity (e.g., Taq DNA polymerase (DNAP), E. coli DNA polymerase I). The cleavage agent may also include modified DNA polymerases having 5′ nuclease activity but lacking synthetic activity. Examples of cleavage agents suitable for use in the method and kits of the present invention are provided in U.S. Pat. Nos. 5,614,402; 5,795,763; 5,843,669; 6,090,606; PCT Appln. Nos WO 98/23774; WO 02/070755A2; and WO0190337A2, each of which is herein incorporated by reference it its entirety.

The term “probe oligonucleotide,” in regard to an INVADER nucleic acid detection assay, refers to an oligonucleotide that interacts with a target nucleic acid to form a cleavage structure in the presence or absence of an INVADER oligonucleotide. When annealed to the target nucleic acid, the probe oligonucleotide and target form a cleavage structure and cleavage occurs within the probe oligonucleotide.

The term “INVADER oligonucleotide” refers to an oligonucleotide that hybridizes to a target nucleic acid at a location near the region of hybridization between a probe and the target nucleic acid, wherein the INVADER oligonucleotide comprises a portion (e.g., a chemical moiety, or nucleotide-whether complementary to that target or not) that overlaps with the region of hybridization between the probe and target. In some embodiments, the INVADER oligonucleotide contains sequences at its 3′ end that are substantially the same as sequences located at the 5′ end of a probe oligonucleotide.

The term “cassette” as used herein refers to an oligonucleotide or combination of oligonucleotides configured to generate a detectable signal in response to cleavage of a probe oligonucleotide in an INVADER assay. In preferred embodiments, the cassette hybridizes to a non-target cleavage product from cleavage of the probe oligonucleotide to form a second invasive cleavage structure, such that the cassette can then be cleaved.

In some embodiments, the cassette is a single oligonucleotide comprising a hairpin portion (i.e., a region wherein one portion of the cassette oligonucleotide hybridizes to a second portion of the same oligonucleotide under reaction conditions, to form a duplex). In other embodiments, a cassette comprises at least two oligonucleotides comprising complementary portions that can form a duplex under reaction conditions. In preferred embodiments, the cassette comprises a label. In particularly preferred embodiments, cassette comprises labeled moieties that produce a fluorescence resonance energy transfer (FRET) effect.

The term “nucleotide analog” as used herein refers to modified or non-naturally occurring nucleotides including but not limited to analogs that have altered stacking interactions such as 7-deaza purines (i.e., 7-deaza-dATP and 7-deaza-dGTP); base analogs with alternative hydrogen bonding configurations (e.g., such as Iso-C and Iso-G and other non-standard base pairs described in U.S. Pat. No. 6,001,983 to S. Benner); non-hydrogen bonding analogs (e.g., non-polar, aromatic nucleoside analogs such as 2,4-difluorotoluene, described by B. A. Schweitzer and E. T. Kool, J. Org. Chem., 1994, 59, 7238-7242, B. A. Schweitzer and E. T. Kool, J. Am. Chem. Soc., 1995, 117, 1863-1872); “universal” bases such as 5-nitroindole and 3-nitropyrrole; and universal purines and pyrimidines (such as “K” and “P” nucleotides, respectively; P. Kong, et al., Nucleic Acids Res., 1989, 17, 10373-10383, P. Kong et al., Nucleic Acids Res., 1992, 20, 5149-5152). Nucleotide analogs include comprise modified forms of deoxyribonucleotides as well as ribonucleotides. The nucleic acid sequences of the present invention may include one or more nucleotide analogs.

The term “sample” in the present specification and claims is used in its broadest sense. On the one hand it is meant to include a specimen or culture (e.g., microbiological cultures). On the other hand, it is meant to include both biological and environmental samples. A sample may include a specimen of synthetic origin.

Biological samples may be animal, including human, fluid, solid (e.g., stool) or tissue, as well as liquid and solid food and feed products and ingredients such as dairy items, vegetables, meat and meat by-products, and waste. Biological samples may be obtained from all of the various families of domestic animals, as well as feral or wild animals, including, but not limited to, such animals as ungulates, bear, fish, lagamorphs, rodents, etc.

Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DESCRIPTION OF THE DRAWINGS

FIG. 1 shows a sequence alignment of most of the sequence of the 5′ UTR of HCV. In particular, an alignment of the following sequences is shown: i) an HCV-1 consensus sequence; ii) a representative HCV-1a sequence (accession number NC-004102); iii) a representative HCV-1b sequence (accession number M58335); iv) sequenced patient sample MPM; v) sequence patient sample LBVA; and yl) sequence of new HCV-1 sub-type HCV-1twt.

FIG. 2A shows an INVADER assay design for detecting an A at position −166 of HCV-1wt, including an INVADER oligonucleotide (SEQ ID NO:20) and a primary probe (SEQ ID NO:21). FIG. 2B shows an INVADER assay design for detecting a G at position −119 of HCV-1twt, including an INVADER oligonucleotide (SEQ ID NO:36) and a primary probe (SEQ ID NO:37).

### DESCRIPTION OF THE INVENTION

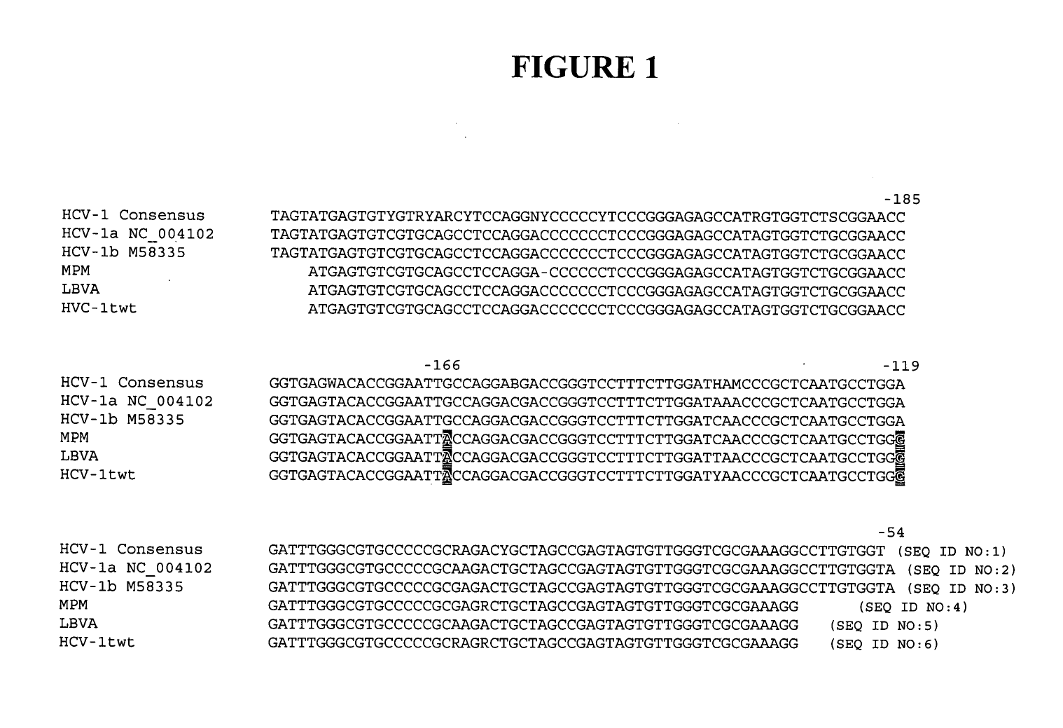

The present invention provides methods and compositions for detecting hepatitis C virus (HCV). In particular, the present invention provides nucleic acid detection assays configured to detect a novel subtype of HCV-1. The novel HCV-1 subtype can be referred to as HCV-1twt. HCV-1twt contains an adenine at position −166 and a guanidine at position −119 as numbered in FIG. 1.

A majority of the 5′ UTR of HCV-1twt is shown in FIG. 1 as SEQ ID NO:6. FIG. 1 also shows the majority of 5′ UTR sequence from two patient samples (MPM and LBVA) found to be infected with HCV. The HCV in these samples were determined to be HCV-1 based on homology to other HCV-1 sequences. Both patients samples were identified as containing new HCV-1 subtype HCV-1twt based on the presence of an adenine at position −166 and a guanidine at position −119.

Tables 1 and 2 below give a number of exemplary probe sequences that may be used to detect the presence of HCV-1twt (e.g. to detect an adenine at position −166 and/or a guanidine at position −119). It is noted that the sequences in these tables are merely exemplary. One of skill in the art could employ similar sequences in order to detect HCV-1twt. For example, one could perform experiments with samples known to contain HCV-1twt to determine if a given probe were useful for detecting HCV-1twt. It is noted that any type of detection may be used, including both direct and indirect detection.

TABLE 1

Exemplary Probes for Detecting A
at Position −166

| | SEQ ID |
|---|---|
| Probe Sequence | NO |
| 5-GGTGAGTACACCGGAATTACCAGGACGACCGGGTCCT-3 | SEQ ID |
| | NO:7 |
| 3-CCACTCATGTGGCCTTAATGGTCCTGCTGGCCCAGGA-5 | SEQ ID |
| | NO:8 |
| 5-TACACCGGAATTACCAGGACGACCG-3 | SEQ ID |
| | NO:9 |
| 3-ATGTGGCCTTAATGGTCCTGCTGGC-5 | SEQ ID |
| | NO:10 |
| 5-GAATTACCAGGACGA-3 | SEQ ID |
| | NO:11 |
| 3-CTTAATGGTCCTGCT-5 | SEQ ID |
| | NO:12 |
| 5-TANACCGNAATTACCAGGACNAC-3 | SEQ ID |
| | NO:13 |
| 3-ATNTGGCNTTAATGGTCCTGNTG-5 | SEQ ID |
| | NO:14 |
| 5-TACCAGGACGACC-3 | SEQ ID |
| | NO:15 |
| 5-ATTACCAGGACGAC-3 | SEQ ID |
| | NO:16 |
| 5-GAATTACCAGGACGA-3 | SEQ ID |
| | NO:17 |
| 5-ACCGGAATTACCAG-3 | SEQ ID |
| | NO:18 |
| 5-CACCGGAATTACC-3 | SEQ ID |
| | NO:19 |
| 5-tttgggttgccTCCgAGAAAGGgCCCGGTtGTCCTGGa-3 | SEQ ID |
| | NO:20 |
| 5-acggacgcggagTAATTCCGaTGTACTCgCCGGT-3 | SEQ ID |
| | NO:21 |

TABLE 2
Exemplary Probes for Detecting G at Position −119

| | |
|---|---|
| Probe Sequence | SEQ ID NO |

| | |
|---|---|
| 5-TYAACCCGCTCAATGCCTGGGGATTTGGGCGTGCCCCCGCR-3 | SEQ ID NO:22 |
| 3-ARTTGGGCGAGTTACGGACCCCTAAACCCGCACGGGGGCGY-5 | SEQ ID NO:23 |
| 5-GCTCAATGCCTGGGGATTTGGGCGTGC-3 | SEQ ID NO:24 |
| 3-CGAGTTACGGACCCCTAAACCCGCACG-5 | SEQ ID NO:25 |
| 5-AATGCCTGGGGATTT-3 | SEQ ID NO:26 |
| 3-TTACGGACCCCTAAA-5 | SEQ ID NO:27 |
| 5-GCTNAATGCNTGGGGATTTGGNCGT-3 | SEQ ID NO:28 |
| 3-CGANTTACGNACCCCTAAACCNGCA-5 | SEQ ID NO:29 |
| 5-GGGATTTGGGCG-3 | SEQ ID NO:30 |
| 5-TGGGGATTTGGGCG-3 | SEQ ID NO:31 |
| 5-CTGGGGATTTGGGC-3 | SEQ ID NO:32 |
| 5-CAATGCCTGGGG-3 | SEQ ID NO:33 |
| 5-AATGCCTGGGGAT-3 | SEQ ID NO:34 |
| 5-ATGCCTGGGGATT-3 | SEQ ID NO:35 |
| 5-atacaactccGCAGcCTTGCGaGGGCACGtCCAAATCa-3 | SEQ ID NO:36 |
| 5-acggacgcggagCCCAGGCAcTGAGCGG-3 | SEQ ID NO:37 |

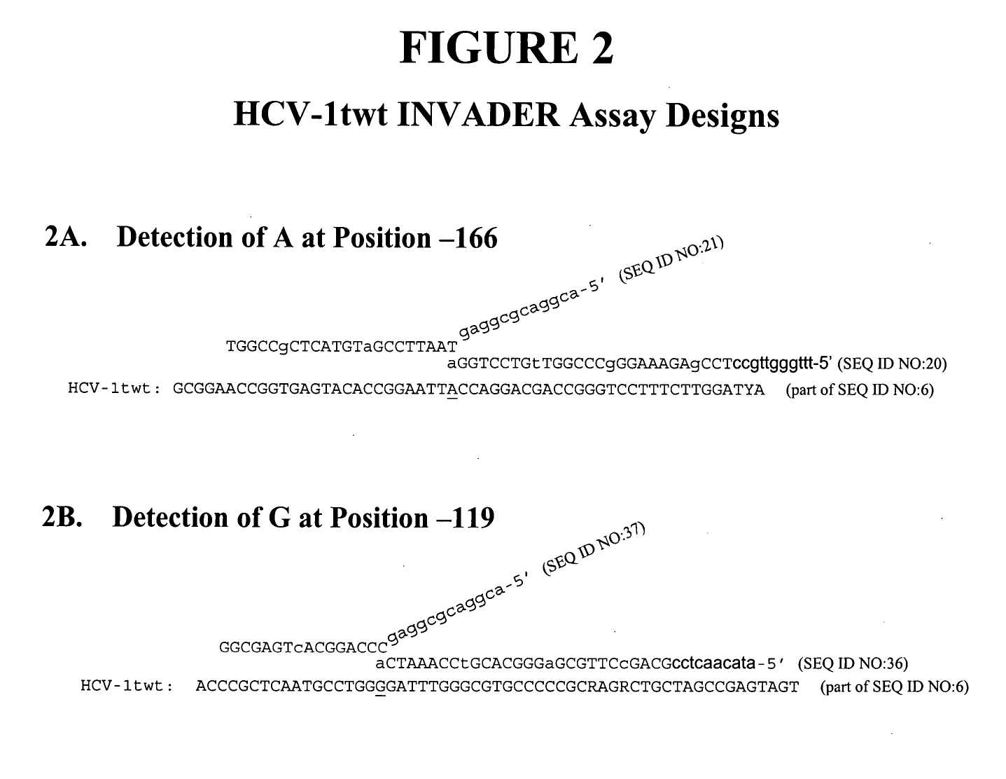

FIGS. 2A and 2B show exemplary INVADER assay designs for detecting HCV-1twt. In particular, FIG. 2A shows an INVADER oligonucleotide (SEQ ID NO:20) and a primary probe (SEQ ID NO:21) arranged in an INVADER assay configuration for detecting an adenine at position −166. FIG. 2B shows an INVADER oligonucleotide (SEQ ID NO:36) and a primary probe (SEQ ID NO:37) arranged in an INVADER assay configuration for detecting a guanidine at position −119. In both of these designs, a structure specific enzyme, such as a thermostable FEN-1 enzyme, can recognize the overlap of both the INVADER oligonucleotide and primary probe at the targeted position (i.e. −166 or −119) and cleave the primary probe such that the presence of an adenine at position −166 or a guanidine at position −119 is detected. Similar INVADER assay designs for detecting positions −166 and −119 can constructed using sequences similar to those shown in FIGS. 2A and 2B. Additional guidance for designing INVADER assays that target these positions of HCV-1twt is found, for example, in U.S. Pat. Nos. 5,846,717; 5,985,557; 5,994,069; 6,001,567; 6,913,881; and 6,090,543, all of which are herein incorporated by reference in their entirities.

In certain embodiments, where an adenine is detected at position −166 of the 5′ UTR of HCV, this indicates to a user that the sample either contains HCV-1twt of HCV-2. In order to help further classify the sample (e.g. definitively establish that HCV-1twt is present in the sample) a second nucleic acid detection assay is employed that can discriminate between HCV-2 and HCV-1twt. In certain embodiments, this second nucleic acid detection assay is configured to detect at least one of the following positions in the 5′ untranslated region: adenine at position −163; cytosine, guanidine, or thymine at position −159; cytosine at position −155; guanidine at position −132; adenine at position −128; thymine at position −122; guanidine or adenine at position −119; guanidine at position −118, thymine at position −80; and cytosine at position −72.

The present invention is not limited by the type of nucleic acid detection assay used to detect bases at positions −166 and −119 in the 5′ UTR of HCV. Detailed below are exemplary nucleic acid detection assays.

1. Direct sequencing Assays

In some embodiments of the present invention, positions −166 and −119 in the 5′ UTR of HCV are detected using a direct sequencing technique. In these assays, nucleic acid samples are first isolated from a sample from a subject using any suitable method. In some embodiments, the region of interest is cloned into a suitable vector and amplified by growth in a host cell (e.g., a bacteria). In other embodiments, nucleic acid in the region of interest is amplified using PCR. Following amplification, nucleic acid in the region of interest is sequenced using any suitable method, including but not limited to manual sequencing using radioactive marker nucleotides, or automated sequencing. The results of the sequencing are displayed using any suitable method. The sequence is examined and the presence or absence of adenine at position −166 and/or guanidine at position −119 is determined.

2. PCR Assays

In some embodiments of the present invention, positions −166 and −119 in the 5′ UTR of HCV are detected using a PCR-based assay. In some embodiments, the PCR assay comprises the use of oligonucleotide primers that hybridize only to the HCV-1twt and primers that will not hybridize to HCV-1twt. Both sets of primers are used to amplify a sample of DNA. If only the HCV-1twt primers result in a PCR product, then the patient is infected with HCV-1twt. If only the non-HCV-1twt primers result in a PCR product, then the patient is not infected with HCV-1twt.

3. Fragment Length Polymorphism Assays

In some embodiments of the present invention, positions −166 and −119 in the 5′ UTR of HCV are detected using a fragment length polymorphism assay. In a fragment length polymorphism assay, a unique DNA banding pattern based on cleaving the DNA at a series of positions is generated using an enzyme (e.g., a restriction enzyme or a CLEAVASE enzyme). Nucleic acid fragments from a sample containing a HCV-1 twt will have a different banding pattern than non-HCV-1twt sequences if the enzyme recognition site involves the adenine at position −166 and/or the guanidine at position −119.

4. Hybridization Assays

In certain embodiments of the present invention, positions −166 and −119 in the 5′ UTR of HCV are detected with a hybridization assay. In a hybridization assay, the presence of absence of adenine at position −166 and/or guanidine at position −119 may be determined based on the ability of the nucleic acid from the sample to hybridize to a complementary nucleic acid molecule (e.g., am oligonucleotide probe, such as those shown in Tables 1 and 2). A variety of exemplary hybridization assays using a variety of technologies for hybridization and detection are described below.

a. Direct Detection of Hybridization

In some embodiments, hybridization of a probe to the sequence of interest is detected directly by visualizing a bound probe (e.g., a Northern or Southern assay; See e.g., Ausabel et al. (eds.), Current Protocols in Molecular Biology, John Wiley & Sons, NY [1991]). In these assays, nucleic acid is isolated from a sample. The DNA or RNA is then separated (e.g., on an agarose gel) and transferred to a membrane. A labeled (e.g., by incorporating a radionucleotide) probe or probes specific for positions −166 and −119 in the 5′ UTR of HCV is allowed to contact the membrane under a condition or low, medium, or high stringency conditions. Unbound probe is removed and the presence of binding is detected by visualizing the labeled probe.

b. Detection of Hybridization Using “DNA Chip” Assays

In some embodiments of the present invention, positions −166 and −119 in the 5′ UTR of HCV are detected using a DNA chip hybridization assay. In this assay, a series of oligonucleotide probes are affixed to a solid support. The oligonucleotide probes are designed to be unique to a given sequence. The DNA sample of interest is contacted with the DNA “chip” and hybridization is detected.

In some embodiments, the DNA chip assay is a GeneChip (Affymetrix, Santa Clara, Calif.; See e.g., U.S. Pat. Nos. 6,045,996; 5,925,525; and 5,858,659; each of which is herein incorporated by reference) assay. The GeneChip technology uses miniaturized, high density arrays of oligonucleotide probes affixed to a “chip.” Probe arrays are manufactured by Affymetrix's light directed chemical synthesis process, which combines solid phase chemical synthesis with photolithographic fabrication techniques employed in the semiconductor industry. Using a series of photolithographic masks to define chip exposure sites, followed by specific chemical synthesis steps, the process constructs high density arrays of oligonucleotides, with each probe in a predefined position in the array. Multiple probe arrays are synthesized simultaneously on a large glass wafer. The wafers are then diced, and individual probe arrays are packaged in injection molded plastic cartridges, which protect them from the environment and serve as chambers for hybridization.

The nucleic acid to be analyzed is isolated, amplified by PCR, and labeled with a fluorescent reporter group. The labeled DNA is then incubated with the array using a fluidics station. The array is then inserted into the scanner, where patterns of hybridization are detected. The hybridization data are collected as light emitted from the fluorescent reporter groups already incorporated into the target, which is bound to the probe array. Probes that perfectly match the target generally produce stronger signals than those that have mismatches. Since the sequence and position of each probe on the array are known, by complementarity, the identity of the target nucleic acid applied to the probe array can be determined.

In other embodiments, a DNA microchip containing electronically captured probes (Nanogen, San Diego, Calif.) is utilized (See e.g., U.S. Pat. Nos. 6,017,696; 6,068,818; and 6,051,380; each of which are herein incorporated by reference). Through the use of microelectronics, Nanogen's technology enables the active movement and concentration of charged molecules to and from designated test sites on its semiconductor microchip. DNA capture probes unique to a given SNP or mutation are electronically placed at, or “addressed” to, specific sites on the microchip. Since DNA has a strong negative charge, it can be electronically moved to an area of positive charge.

In still further embodiments, an array technology based upon the segregation of fluids on a flat surface (chip) by differences in surface tension (ProtoGene, Palo Alto, Calif.) is utilized (See e.g., U.S. Pat. Nos. 6,001,311; 5,985,551; and 5,474,796; each of which is herein incorporated by reference). Protogene's technology is based on the fact that fluids can be segregated on a flat surface by differences in surface tension that have been imparted by chemical coatings. Once so segregated, oligonucleotide probes are synthesized directly on the chip by ink jet printing of reagents. The array with its reaction sites defined by surface tension is mounted on a X/Y translation stage under a set of four piezoelectric nozzles, one for each of the four standard DNA bases. The translation stage moves along each of the rows of the array and the appropriate reagent is delivered to each of the reaction site. For example, the A amidite is delivered only to the sites where amidite A is to be coupled during that synthesis step and so on. Common reagents and washes are delivered by flooding the entire surface and then removing them by spinning.

DNA probes unique for positions −166 and −119 in the 5′ UTR of HCV are affixed to the chip using Protogene's technology. The chip is then contacted with the sample potentially containing HCV-1twt. Following hybridization, unbound DNA is removed and hybridization is detected using any suitable method (e.g., by fluorescence de-quenching of an incorporated fluorescent group).

In yet other embodiments, a “bead array” is used for the detection of adenine at position −166 and/or guanidine at position −119 in the 5′ UTR of HCV (Illumina, San Diego, Calif.; See e.g., PCT Publications WO 99/67641 and WO 00/39587, each of which is herein incorporated by reference). Illumina uses a BEAD ARRAY technology that combines fiber optic bundles and beads that self assemble into an array. Each fiber optic bundle contains thousands to millions of individual fibers depending on the diameter of the bundle. The beads are coated with an oligonucleotide specific for HCV-1twt. Batches of beads are combined to form a pool specific to the array. To perform an assay, the BEAD ARRAY is contacted with a prepared subject sample (e.g., DNA). Hybridization is detected using any suitable method.

c. Enzymatic Detection of Hybridization

In some embodiments of the present invention, hybridization is detected by enzymatic cleavage of specific structures (e.g., INVADER assay, Third Wave Technologies; See e.g., U.S. Pat. Nos. 5,846,717; 5,985,557; 5,994,069; 6,001,567; 6,913,881; and 6,090,543, WO 97/27214, WO 98/42873, Lyamichev et al., Nat. Biotech., 17:292 (1999), Hall et al., PNAS, USA, 97:8272 (2000), each of which is herein incorporated by reference in their entirety for all purposes). The INVADER assay detects specific DNA and RNA sequences by using structure specific enzymes to cleave a complex formed by the hybridization of overlapping oligonucleotide probes. Elevated temperature and an excess of one of the probes enable multiple probes to be cleaved for each target sequence present without temperature cycling. These cleaved probes then direct cleavage of a second labeled probe. The secondary probe oligonucleotide can be 5′ end labeled with a fluorescent dye that is quenched by a second dye or other quenching moiety. Upon cleavage, the de-quenched dye-labeled product may be detected using a standard fluorescence plate reader, or an instrument configured to collect fluorescence data during the course of the reaction (i.e., a “real-time” fluorescence detector, such as an ABI 7700 Sequence Detection System, Applied Biosystems, Foster City, Calif.).

In an embodiment of the INVADER assay used for detecting SNPs, such as those at positions −166 and −119 of the 5′ UTR of HCV, two oligonucleotides (a primary probe specific either for a particular base at the SNP, and an INVADER oligonucleotide) hybridize in tandem to the target nucleic acid to form an overlapping structure. A structure-specific nuclease enzyme recognizes this overlapping structure and cleaves the primary probe. In a secondary reaction, cleaved primary probe combines with a fluorescence-labeled secondary probe to create another overlapping structure that is cleaved by the enzyme. The initial and secondary reactions can run concurrently in the same vessel. Cleavage of the secondary probe is detected by using a fluorescence detector, as described above. The signal of the test sample may be compared to known positive and negative controls.

5. Other Detection Assays

Additional detection assays that are produced and utilized using the systems and methods of the present invention include, but are not limited to, enzyme mismatch cleavage methods (e.g., Variagenics, U.S. Pat. Nos. 6,110,684, 5,958,692, 5,851,770, herein incorporated by reference in their entireties); polymerase chain reaction; branched hybridization methods (e.g., Chiron, U.S. Pat. Nos. 5,849,481, 5,710,264, 5,124,246, and 5,624,802, herein incorporated by reference in their entireties); rolling circle replication (e.g., U.S. Pat. Nos. 6,210,884 and 6,183,960, herein incorporated by reference in their entireties); NASBA (e.g., U.S. Pat. No. 5,409,818, herein incorporated by reference in its entirety); molecular beacon technology (e.g., U.S. Pat. No. 6,150,097, herein incorporated by reference in its entirety); E-sensor technology (Motorola, U.S. Pat. Nos. 6,248,229, 6,221,583, 6,013,170, and 6,063,573, herein incorporated by reference in their entireties); cycling probe technology (e.g., U.S. Pat. Nos. 5,403,711, 5,011,769, and 5,660,988, herein incorporated by reference in their entireties); Dade Behring signal amplification methods (e.g., U.S. Pat. Nos. 6,121,001, 6,110,677, 5,914,230, 5,882,867, and 5,792,614, herein incorporated by reference in their entireties); ligase chain reaction (Barnay Proc. Natl. Acad. Sci. USA 88, 189-93 (1991)); and sandwich hybridization methods (e.g., U.S. Pat. No. 5,288,609, herein incorporated by reference in its entirety).

6. Mass Spectroscopy Assay

In some embodiments, a MassARRAY system (Sequenom, San Diego, Calif.) is used to detect positions −166 and −119 in the 5′ UTR of HCV (See e.g., U.S. Pat. Nos. 6,043,031; 5,777,324; and 5,605,798; each of which is herein incorporated by reference). DNA is isolated from blood samples using standard procedures. Next, specific DNA regions containing the region of HCV 5′ UTR of interest (e.g, about 200 base pairs in length) are amplified by PCR. The amplified fragments are then attached by one strand to a solid surface and the non immobilized strands are removed by standard denaturation and washing. The remaining immobilized single strand then serves as a template for automated enzymatic reactions that produce genotype specific diagnostic products.

Very small quantities of the enzymatic products, typically five to ten nanoliters, are then transferred to a SpectroCHIP array for subsequent automated analysis with the SpectroREADER mass spectrometer. Each spot is preloaded with light absorbing crystals that form a matrix with the dispensed diagnostic product. The MassARRAY system uses MALDI TOF (Matrix Assisted Laser Desorption Ionization Time of Flight) mass spectrometry. In a process known as desorption, the matrix is hit with a pulse from a laser beam. Energy from the laser beam is transferred to the matrix and it is vaporized resulting in a small amount of the diagnostic product being expelled into a flight tube. As the diagnostic product is charged when an electrical field pulse is subsequently applied to the tube they are launched down the flight tube towards a detector. The time between application of the electrical field pulse and collision of the diagnostic product with the detector is referred to as the time of flight. This is a very precise measure of the product's molecular weight, as a molecule's mass correlates directly with time of flight with smaller molecules flying faster than larger molecules. The entire assay is completed in less than one thousandth of a second, enabling samples to be analyzed in a total of 3-5 second including repetitive data collection. The SpectroTYPER software then calculates, records, compares and reports the genotypes at the rate of three seconds per sample.

7. HPLC and CE Detection

The present invention contemplates detection of HCV nucleic acid by high performance liquid chromatography (HPLC). HPLC generally refers to a technique for partitioning a sample or more specifically the components of a sample between a liquid moving or mobile phase and a solid stationary phase (see, e.g., U.S. Pat. Nos. 6,453,244; 6,642,374; and 6,579,459; all of which are herein incorporated by reference, and all of which describe methods for detecting nucleic acid by HPLC). In certain embodiments, nano-flow HPCL methods are employed to detect HCV nucleic acid. Nano-flow HPLC generally involves very narrow capillaries and small reactions volumes and is a very sensitive detection method.

The present invention also contemplates detecting HCV nucleic acid by capillary electrophoresis (CE). In general, CE referres to modes of separation which harness electrical forces in capillary tubes for analytical purposes. CE underpins modern genomics and is becoming increasingly important in the developing fields of proteomics and metabolite profiling. Extremely high separation efficiencies are routinely obtained with good reproducibility. References describing the use of CE to detect nucleic acids include, but are not limited to, U.S. Pat. Nos. 5,874,213; 6,177,247; and 5,409,586; all of which are herein incorpoared by reference.

All publications and patents mentioned in the above specification are herein incorporated by reference as if expressly set forth herein. Various modifications and variations of the described assays of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the following claims.

We claim:

1. A method comprising contacting a sample suspected of containing hepatitis C virus with a first nucleic acid detection assay under conditions such that the presence of adenine at position −166 of the 5′ untranslated region of said hepatitis C virus is detected, thereby identifying said sample as containing said hepatitis C virus.

2. The method of claim 1, wherein said first nucleic acid detection assay comprises an invasive cleavage detection assay.

3. The method of claim 1, wherein said first nucleic acid detection assay is selected from the group consisting of: a TAQMAN assay, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, a sandwich hybridization assay, and a Line Probe Assay.

4. The method of claim 1, wherein said first nucleic acid detection assay comprises a Line Probe Assay.

5. The method of claim 1, further comprising contacting said sample with a second nucleic acid detection assay configured to detect at least one of the following positions in said 5′ untranslated region: adenine at position −163; cytosine, guanidine, or thymine at position −159; cytosine at position −155; guanidine at position −132; adenine at position −128; thymine at position −122; guanidine or adenine at position −119; guanidine at position −118, thymine at position −80; and cytosine at position −72.

6. The method of claim 5, wherein said first nucleic acid detection assay and said second nucleic acid detection assay together are capable of identifying said hepatitis C virus as HCV-1twt.

7. A method comprising contacting a sample suspected of containing hepatitis C virus with a nucleic acid detection assay under conditions such that the presence of a guanidine at position −119 of the 5′ untranslated region of said hepatitis C virus is detected.

8. The method of claim 7, wherein said nucleic acid detection assay comprises an invasive cleavage detection assay.

9. The method of claim 7, wherein said nucleic acid detection assay is selected from the group consisting of: a TAQMAN assay, a sequencing assay, a polymerase chain reaction assay, a hybridization assay, a microarray assay, a bead array assay, a primer extension assay, an enzyme mismatch cleavage assay, a branched hybridization assay, a rolling circle replication assay, a NASBA assay, a molecular beacon assay, a cycling probe assay, a ligase chain reaction assay, a sandwich hybridization assay, and a Line Probe Assay.

10. The method of claim 7, further comprising contacting said sample with a second nucleic acid detection assay configured to detect at least one of the following positions in said 5′ untranslated region: adenine at position −163; cytosine, guanidine, or thymine at position −159; cytosine at position −155; guanidine at position −132; adenine at position −128; thymine at position −122; guanidine or adenine at position −119; guanidine at position −118, thymine at position −80; and cytosine at position −72.

11. The method of claim 7, wherein said nucleic acid detection assay is capable of identifying said hepatitis C virus as HCV-1twt.

12. A composition comprising an isolated first nucleic acid sequence or an isolated second nucleic acid sequence, wherein said first nucleic acid sequence comprises a sequence selected from the group consisting of SEQ ID NOs:7-21 and SEQ ID NOs:22-37, and wherein said second nucleic acid sequence is configured to hybridize to said first nucleic acid sequence under high stringency conditions.

13. The composition of claim 12, wherein said first and second nucleic acid sequences are both present in said composition.


SEQUENCE LISTING (37 sequences)

SEQ ID NO: 1 - DNA, 196 bp, Artificial Sequence
  Synthetic
  misc_feature (28)..(28): n is a, c, g, or t
tagtatgagtgtygtryarcytccaggnycccccytcccgggagagccatrgtggtctscggaaccggtgagwacaccggaattgccaggabgaccgggtcctttcttggathamcccgctcaatgcctggagatttgggcgtgcccccgcragacygctagccgagtagtgttgggtcgcgaaaggccttgtggt

SEQ ID NO: 2 - DNA, 197 bp, Hepatitis C virus
tagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggta

SEQ ID NO: 3 - DNA, 197 bp, Hepatitis C virus
tagtatgagtgtcgtgcagcctccaggaccccccctcccgggagagccatagtggtctgcggaaccggtgagtacaccggaattgccaggacgaccgggtcctttcttggatcaacccgctcaatgcctggagatttgggcgtgcccccgcgagactgctagccgagtagtgttgggtcgcgaaaggccttgtggta

SEQ ID NO: 4 - DNA, 182 bp, Homo sapiens
atgagtgtcgtgcagcctccaggacccccctcccgggagagccatagtggtctgcggaaccggtgagtacaccggaattaccaggacgaccgggtcctttcttggatcaacccgctcaatgcctggggatttgggcgtgcccccgcgagrctgctagccgagtagtgttgggtcgcgaaagg

SEQ ID NO: 5 - DNA, 183 bp, Homo sapiens
atgagtgtcgtgcagcctccaggaccccccctcccgggagagccatagtggtctgcggaaccggtgagtacaccggaattaccaggacgaccgggtcctttcttggattaacccgctcaatgcctggggatttgggcgtgcccccgcaagactgctagccgagtagtgttgggtcgcgaaagg

SEQ ID NO: 6 - DNA, 183 bp, Hepatitis C virus
atgagtgtcgtgcagcctccaggaccccccctcccgggagagccatagtggtctgcggaaccggtgagtacaccggaattaccaggacgaccgggtcctttcttggatyaacccgctcaatgcctggggatttgggcgtgcccccgcragrctgctagccgagtagtgttgggtcgcgaaagg

SEQ ID NO: 7 - DNA, 37 bp, Artificial Sequence
  Synthetic
ggtgagtacaccggaattaccaggacgaccgggtcct

SEQ ID NO: 8 - DNA, 37 bp, Artificial Sequence
  Synthetic
aggacccggtcgtcctggtaattccggtgtactcacc

SEQ ID NO: 9 - DNA, 25 bp, Artificial Sequence
  Synthetic
tacaccggaattaccaggacgaccg

SEQ ID NO: 10 - DNA, 25 bp, Artificial Sequence
  Synthetic
cggtcgtcctggtaattccggtgta

SEQ ID NO: 11 - DNA, 15 bp, Artificial Sequence
  Synthetic
gaattaccaggacga

SEQ ID NO: 12 - DNA, 15 bp, Artificial Sequence
  Synthetic
tcgtcctggtaattc

SEQ ID NO: 13 - DNA, 23 bp, Artificial Sequence
  Synthetic
  misc_feature (3)..(3): n is a, c, g, or t
  misc_feature (8)..(8): n is a, c, g, or t
  misc_feature (21)..(21): n is a, c, g, or t
tanaccgnaattaccaggacnac

SEQ ID NO: 14 - DNA, 23 bp, Artificial Sequence
  Synthetic
  misc_feature (3)..(3): n is a, c, g, or t
  misc_feature (16)..(16): n is a, c, g, or t
  misc_feature (21)..(21): n is a, c, g, or t
gtngtcctggtaattncggtnta

SEQ ID NO: 15 - DNA, 13 bp, Artificial Sequence
  Synthetic
taccaggacgacc

SEQ ID NO: 16 - DNA, 14 bp, Artificial Sequence
  Synthetic
attaccaggacgac

SEQ ID NO: 17 - DNA, 15 bp, Artificial Sequence
  Synthetic
gaattaccaggacga

SEQ ID NO: 18 - DNA, 14 bp, Artificial Sequence
  Synthetic
accggaattaccag

SEQ ID NO: 19 - DNA, 13 bp, Artificial Sequence
  Synthetic
caccggaattacc

SEQ ID NO: 20 - DNA, 38 bp, Artificial Sequence
  Synthetic
tttgggttgcctccgagaaagggcccggttgtcctgga

SEQ ID NO: 21 - DNA, 34 bp, Artificial Sequence
  Synthetic
acggacgcggagtaattccgatgtactcgccggt

SEQ ID NO: 22 - DNA, 41 bp, Artificial Sequence
  Synthetic
tyaacccgctcaatgcctggggatttgggcgtgcccccgcr

SEQ ID NO: 23 - DNA, 41 bp, Artificial Sequence
  Synthetic
ygcgggggcacgcccaaatccccaggcattgagcgggttra

SEQ ID NO: 24 - DNA, 27 bp, Artificial Sequence
  Synthetic
gctcaatgcctggggatttgggcgtgc

SEQ ID NO: 25 - DNA, 27 bp, Artificial Sequence
  Synthetic
gcacgcccaaatccccaggcattgagc

SEQ ID NO: 26 - DNA, 15 bp, Artificial Sequence
  Synthetic
aatgcctggggattt

SEQ ID NO: 27 - DNA, 15 bp, Artificial Sequence
  Synthetic
aaatccccaggcatt

SEQ ID NO: 28 - DNA, 25 bp, Artificial Sequence
  Synthetic
  misc_feature (4)..(4): n is a, c, g, or t
  misc_feature (10)..(10): n is a, c, g, or t
  misc_feature (22)..(22): n is a, c, g, or t
gctnaatgcntggggatttggncgt

SEQ ID NO: 29 - DNA, 25 bp, Artificial Sequence
  Synthetic
  misc_feature (4)..(4): n is a, c, g, or t
  misc_feature (16)..(16): n is a, c, g, or t
  misc_feature (22)..(22): n is a, c, g, or t
acgnccaaatccccangcattnagc

SEQ ID NO: 30 - DNA, 12 bp, Artificial Sequence
  Synthetic
gggatttgggcg

SEQ ID NO: 31 - DNA, 14 bp, Artificial Sequence
  Synthetic
tggggatttgggcg

SEQ ID NO: 32 - DNA, 14 bp, Artificial Sequence
  Synthetic
ctggggatttgggc

SEQ ID NO: 33 - DNA, 12 bp, Artificial Sequence
  Synthetic
caatgcctgggg

SEQ ID NO: 34 - DNA, 13 bp, Artificial Sequence
  Synthetic
aatgcctggggat

SEQ ID NO: 35 - DNA, 13 bp, Artificial Sequence
  Synthetic
atgcctggggatt

SEQ ID NO: 36 - DNA, 38 bp, Artificial Sequence
  Synthetic
atacaactccgcagccttgcgagggcacgtccaaatca

SEQ ID NO: 37 - DNA, 28 bp, Artificial Sequence
  Synthetic
acggacgcggagcccaggcactgagcgg